(19) United States
(12) Patent Application Publication
(10) Pub. No.: US 2015/0257942 A1
(43) Pub. Date: Sep. 17, 2015

(54) MONITORING SYSTEM FOR INFANT AND PATIENTS WITH ADVANCED DISEASE FOR AUTOMATICALLY ALERTING OF URINE AND FECES

(71) Applicants: Donggyu KIM, (US); Chigeon CHOI, Haeundae-gu Busan (KR)

(72) Inventors: Donggyu Kim, Busan (KR); Chigeon Choi, Busan (KR)

(21) Appl. No.: 14/433,625

(22) PCT Filed: Oct. 4, 2012
(86) PCT No.: PCT/KR2012/008002

§ 371 Date: Apr. 3, 2015

(51) Int. Cl. A61F 13/42; G08B 25/10

(52) U.S. Cl. CPC A61F 13/42; G08B 25/10; A61F 2013/421; A61F 2013/424

(57) ABSTRACT

The present invention comprises: a diaper cover into which a diaper is inserted; a sensing pad insertively fixed into the diaper cover and of which an upper portion comprises a feces sensing means and a urine sensing means which come in contact with the diaper so as to sense the urine and feces state of the diaper; and a state display portion which is electrically connected to the sensing pad and receives the urine and feces state of the diaper sensed at the sensing pad so as to display the same, wherein the feces sensing means and the urine sensing means comprise a hydrogen sensor, a carbon dioxide sensor, a humidity sensor and temperature sensor.

### TECHNICAL FIELD

The present invention relates to a monitoring system for automatically alerting urine and feces of infants and critical patients. More particularly, the present invention relates to a monitoring system for automatically alerting urine and feces of infants and critical patients, in which a temperature sensor, a humidity sensor, a hydrogen sensor, and a carbon dioxide sensor are mounted in a sensing pad to accurately sense the presence or absence of urine and feces, a concentration sensor is further provided to easily measure the amount of feces based on the amount of carbon dioxide, a state display unit and a wall pad are connected via wireless communication to facilitate the installation and management of the system, the patient's name, image, change in the amount of feces, etc. are displayed on the wall pad to allow a manager to rapidly and easily manage the patients, a program which can modify the input is operated on a server to calculate the medical history, treatment history, personal data, and changes in the frequency and amount of urine and feces of a patient, from which urine and feces are sensed, and display the information in graphs through an image output unit such that the patient can be provided with a clean diaper at all times and the manager can easily, reliably and efficiently manage the patients, and a virus piezoelectric element is used as a power source to drive the sensors and a microcontroller to reduce the electricity cost and protect the environment.

### BACKGROUND ART

Generally, a cloth diaper has been used to prevent urine excreted from a user wearing the diaper from leaking to the outside. Examples of functional diapers that have appeared since then include a disposable diaper that is discolored when a user wearing the diaper urinates to indicate the presence or absence of urine to the outside and a diaper cover that is attachable to a diaper and includes a sensing pad that is attached to or mounted in the diaper cover and configured to indicate the presence or absence of urine and feces to the outside in an alarming manner or wireless manner using a sensor.

However, these conventional techniques have not provided technical means to detect urine and feces by means other than the sensor and thus easily respond to even a little urine and feces. Moreover, the effectiveness of the techniques is reduced due to high power consumption, and there is no suitable device to absorb any leaks from the diaper, which is problematic.

Accordingly, in order to solve these problems, the present applicant has invented “a reusable automatic device for sensing urine and feces” and registered a patent (Korean Patent No.: 0995966).

According to this patent, information about urine and feces in a diaper worn by a patient is collected by a sensing pad that does not use expensive sensors, and the collected information is provided on a state display unit, thus reducing the production cost and ensuring the ease of use.

However, the application of the sensing device of the above patent to actual products results in many problems that the accuracy is somewhat reduced because the presence or absence of urine and feces is sensed by a temperature sensor and a humidity sensor, the installation and management of the system is troublesome because the sensed information about urine and feces is transmitted over wires, the battery used as a power source to operate the sensors should be replaced frequently, which increases the cost, it is difficult for a medical team to match and determine patient information, urine and feces information, and diagnosis and treatment details, and it is difficult to determine the amount of urine and feces.

### DISCLOSURE

### Technical Problem

The present invention has been made in an effect to solve the above-described problems, and an object of the present invention is to provide a monitoring system for automatically alerting urine and feces of infants and critical patients which can accurately sense the presence or absence of urine and feces, can easily measure the amount of feces, simplifies the installation and management of the system, allows a manager to rapidly and easily manage the patients, allows the patient to be provided with a clean diaper at all times, allows the manager to easily, reliably and efficiently manage the patients, and can reduce the electricity cost and protect the environment.

### Technical Solution

To achieve the above-described object, a monitoring system for automatically alerting urine and feces of infants and critical patients according to the present invention comprises: a diaper cover into which a diaper is introduced; a sensing pad into which the diaper cover is inserted and fixed and which comprises a feces sensing means and a urine sensing means to sense a urine and feces state of the diaper when the top of the sensing pad comes in contact with the diaper; and a state display unit which is electrically connected to the sensing pad to receive and display information on the urine and feces state of the diaper sensed by the sensing pad, in which each of the feces sensing means and the urine sensing means comprises a hydrogen sensor, a carbon dioxide sensor, a humidity sensor, and a temperature sensor.

Moreover, the monitoring system may further comprise a power source to drive the hydrogen sensor, the carbon dioxide sensor, the humidity sensor, and the temperature sensor, and the power source may comprise a piezoelectric element in which viruses with helical proteins are stacked on the surface thereof.

Furthermore, the sensing pad may further comprise a concentration sensor which measures the amount of feces by measuring the amount of hydrogen and carbon dioxide sensed by the hydrogen sensor and the carbon dioxide sensor.

In addition, the state display unit may be mounted on a patient's bed, the monitoring system may further comprise a wall pad which is connected to the state display unit via wireless communication and located on the wall of a patient room, and the wall pad may display identification information of a patient, from which urine and feces are sensed, including the patient's bed number, name, and image.

Additionally, the monitoring system may further comprise a multiport, a server, and an image output unit, in which the multiport is connected to the wall pad via wire communication to gather diaper state information through a serial communication line, and the server is electrically connected to the multiport to calculate the medical history, treatment history, personal data, and changes in the frequency and amount of urine and feces of a patient, from which urine and feces are sensed, and display the information in graphs through the image output unit.

### DESCRIPTION OF DRAWINGS

FIG. 1 is a perspective view showing a diaper cover, a sensing pad, and a state display unit in a monitoring system for automatically alerting urine and feces of infants and critical patients in accordance with an embodiment of the present invention.

FIG. 2 is a conceptual view showing the entire configuration of a monitoring system for automatically alerting urine and feces of infants and critical patients in accordance with an embodiment of the present invention.

FIG. 3 is a perspective view showing that the monitoring system of FIG. 2 is applied to actual patient rooms.

FIG. 4 is a circuit diagram of a sensing pad, a state display unit, and a wall pad in FIG. 3.

Description of Reference Numerals for Main Parts in the Drawings:

| | |
|---|---|
| 5: wear part | 10: diaper cover |
| 20: sensing pad | 21: urine sensing means |
| 22: feces sensing means | 50: state display unit |
| 60: wall pad | 70: multiport |
| 75: server | 80: image output unit |

### EMBODIMENTS FOR CARRYING OUT THE INVENTION

The monitoring system for automatically alerting urine and feces of infants and critical patients according to the present invention comprises: a diaper cover into which a diaper is introduced; a sensing pad into which the diaper cover is inserted and fixed and which comprises a feces sensing means and a urine sensing means to sense a urine and feces state of the diaper when the top of the sensing pad comes in contact with the diaper; and a state display unit which is electrically connected to the sensing pad to receive and display information on the urine and feces state of the diaper sensed by the sensing pad, in which each of the feces sensing means and the urine sensing means comprises a hydrogen sensor, a carbon dioxide sensor, a humidity sensor, and a temperature sensor.

### MODE FOR INVENTION

Hereinafter, embodiments of the present invention will be described in more detail with reference to the accompanying drawings. It should be noted that in assigning reference numerals to components throughout the drawings, like reference numerals designate like components even though components are shown in different drawings. Moreover, in describing the present invention, a detailed description of related known functions or configurations will be omitted so as not to obscure the subject of the present invention. Furthermore, the embodiments of the present invention will be described below, but the technical idea of the present invention is not limited or restricted thereto and can be modified and carried out in various manners by those skilled in the art.

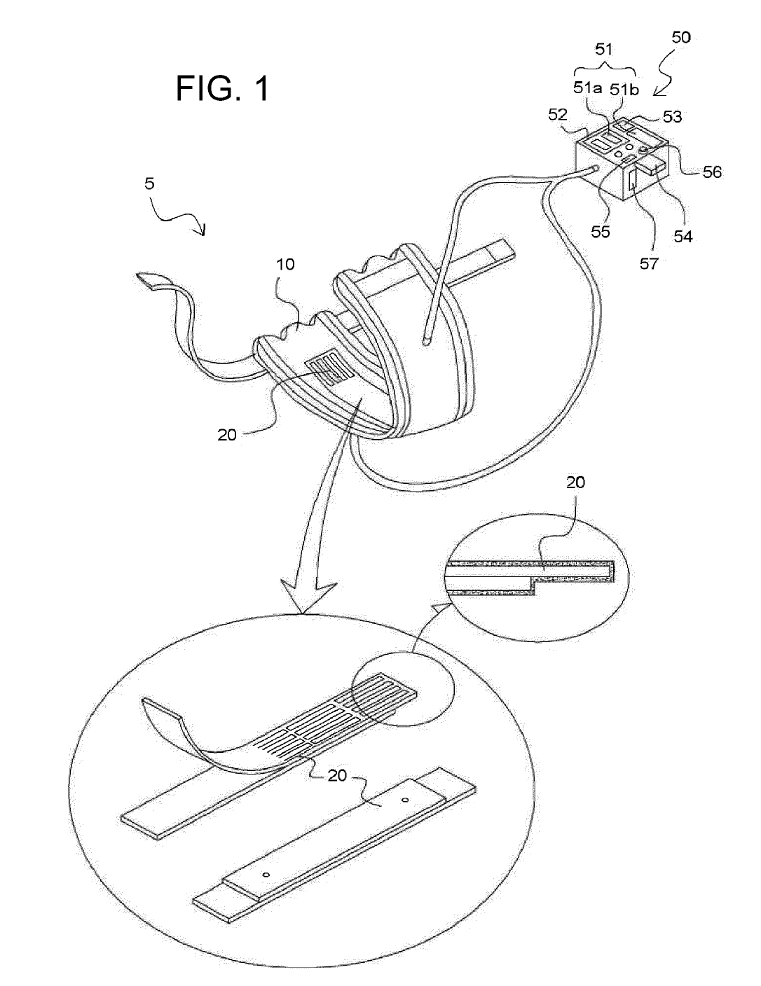

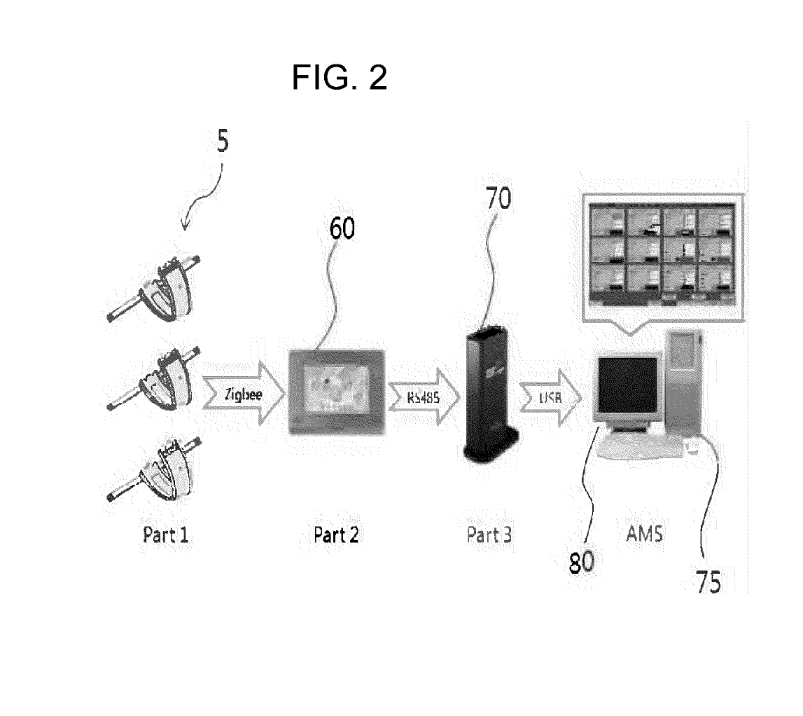

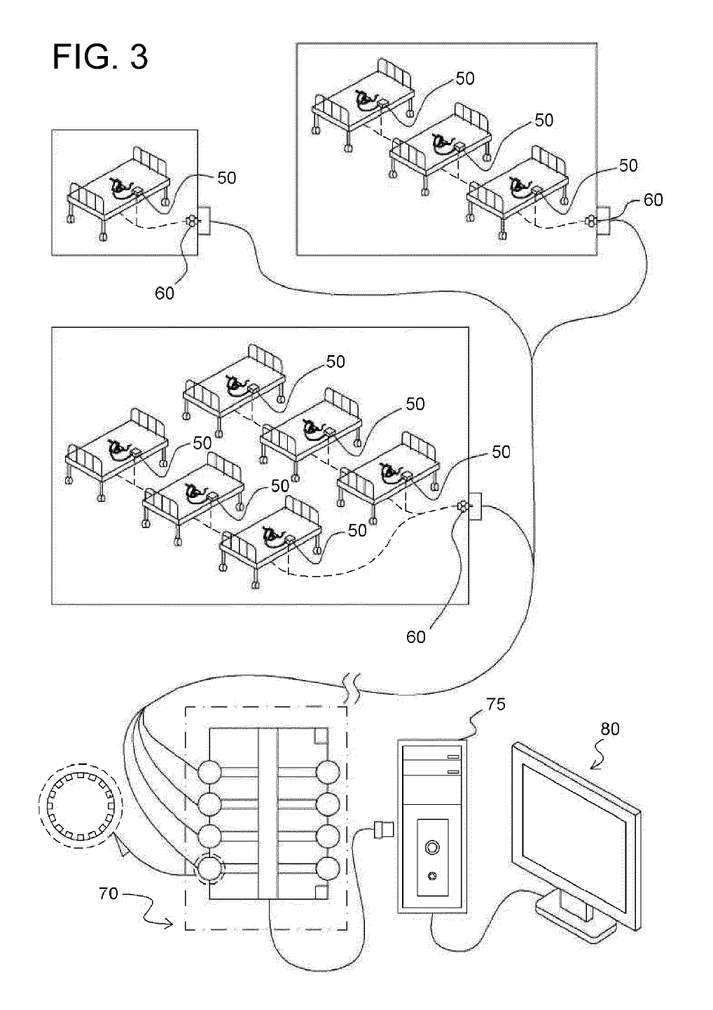

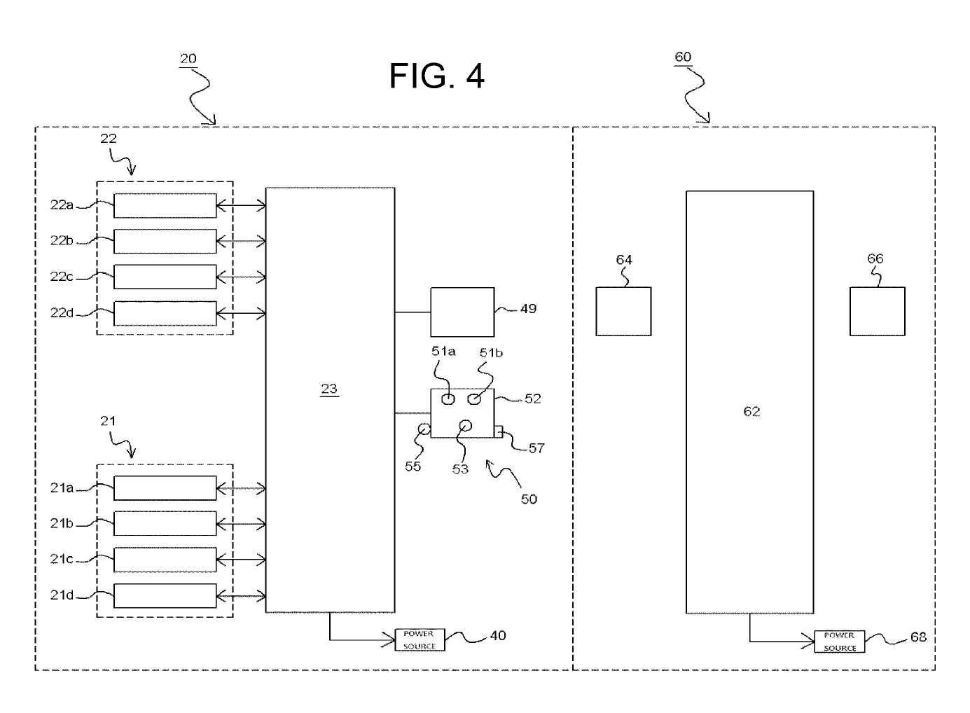

FIG. 1 is a perspective view showing a diaper cover, a sensing pad, and a state display unit in a monitoring system for automatically alerting urine and feces of infants and critical patients in accordance with an embodiment of the present invention. FIG. 2 is a conceptual view showing the entire configuration of a monitoring system for automatically alerting urine and feces of infants and critical patients in accordance with an embodiment of the present invention. FIG. 3 is a perspective view showing that the monitoring system of FIG. 2 is applied to actual patient rooms. FIG. 4 is a circuit diagram of a sensing pad, a state display unit, and a wall pad in FIG. 3.

A monitoring system for automatically alerting urine and feces of infants and critical patients in accordance with an embodiment of the present invention comprises a wear part 5, a state display unit 50, a wall pad 60, a multiport 70, a server 75, and an image output unit 80, as shown in FIGS. 1 to 4.

The wear part 5 comprises a diaper cover 10 and a sensing pad 20.

The diaper cover 10 has a U shape such that a diaper (not shown) is introduced into the inside thereof, and an insertion groove is provided on the inner surface in the longitudinal direction.

The sensing pad 20 is inserted and fixed to the insertion groove of the diaper cover 10 and comprises a urine sensing means 21, a feces sensing means 22, and a microcontroller 23 to sense a urine and feces state of the diaper when the top of the sensing pad 20 comes in contact with the diaper.

The urine sensing means 21 comprises a temperature sensor 21a, a humidity sensor 21b, a hydrogen sensor 21c, and a carbon dioxide sensor 21d. The urine sensing means 21 senses the temperature rise by means of the temperature sensor 21a when moisture from the urine present in the diaper is sensed by the humidity sensor 21b, and the sensed signal is displayed as a urine state by a urine sensing lamp 51a of the state display unit 50 electrically connected to the sensing pad 20.

The feces sensing means 22 comprises a temperature sensor 22a, a humidity sensor 22b, a hydrogen sensor 22c, and a carbon dioxide sensor 22d.

During bowel movement, ammonia (NH₃), an intestinal gas, comes from intestines, and even when feces are not expelled, ammonia is excreted from the body through a fart. However, this ammonia is not easy to measure, which is not easy to be sensed by a sensor.

Therefore, in the present invention, the hydrogen (H₂) sensor and the carbon dioxide (CO₂) sensor are used to determine the discharge of intestinal gases. In a standby state where the diaper is opened, the presence and amount of hydrogen is not sensed, which makes it difficult to measure the hydrogen, but in a closed state where the diaper is worn, the presence and amount of hydrogen can be sensed, which makes it possible to measure the hydrogen.

The hydrogen sensor 22c and the carbon dioxide sensor 22d determine the presence or absence of feces by sensing the presence of hydrogen and carbon dioxide, intestinal gases, generated when a patient wearing the diaper excretes feces. Meanwhile, the humidity sensor 22b for sensing the humidity of feces and the temperature sensor 22a for sensing the temperature of feces are provided to determine the presence or absence of feces, and the sensed signal is displayed as a feces state by a feces sensing lamp 51b of the state display unit 50 electrically connected to the sensing pad 20.

At this time, even when the presence of hydrogen and carbon dioxide is sensed by the hydrogen sensor 22c and the carbon dioxide sensor 22d, if the heat and humidity are not sensed by the temperature sensor 22a and the humidity sensor 22b, it is determined that the intestinal gases are discharged to the outside through a fart, not as a feces state.

Although not shown, the urine sensing means 21 and the feces sensing means 22 may further comprise a concentration sensor. The concentration sensor is used to measure the amount of feces by measuring the amount of hydrogen and carbon dioxide sensed by the hydrogen sensor and the carbon dioxide sensor. Therefore, the amount of feces can be measured without having to mount a separate weight sensor.

The microcontroller 23 controls the operation of the urine sensing means 21 and the feces sensing means 22 and stores information about the sensed urine and feces.

A power source 40 is provided to drive the various sensors and the microcontroller. The power source 40 generates power by means of a piezoelectric element in which viruses with helical proteins are stacked on the surface thereof.

Currently used piezoelectric elements contain substances harmful to humans such as lead or zirconium, and the manufacturing process thereof is complex. On the contrary, viruses such as M13 viruses are harmless to humans. The M13 virus has protein strands coiled helically on the surface thereof, and when these helical protein strands are pushed or twisted, both ends are charged with positive (+) electricity and negative (−) electricity, respectively, thus generating current.

M13 viruses are stacked on the surface of a virus piezoelectric element, and electrodes of gold (Au), for example, are connected to the top and bottom thereof to maximize the efficiency of the virus piezoelectric element and the electric power generation. Viruses have self assembly capability, and thus when a film is immersed in a solution containing viruses, the rod-shaped viruses are evenly distributed even without applying a force from the outside. Therefore, the piezoelectric element is produced by stacking viruses in the above manner.

The state display unit 50 is electrically connected to the sensing pad 20 to receive and display information on the urine and feces state of the diaper sensed by the sensing pad 20. The state display unit 50 comprises the urine sensing lamp 51a, the feces sensing lamp 51b, a liquid crystal display 52, a speaker 53, a selection switch 55, and an on/off switch 57. For example, the state display unit 50 may be mounted on a patient bed as shown in FIG. 3.

The state display unit 50 comprises a transceiver 49 to enable wireless communication with the wall pad 60. The urine and feces state based on the sensed values of the humidity sensors is displayed in graphs on the liquid crystal display 52 to allow a manager to determine a patient's diaper state information in more detail.

When urine and feces are sensed by the sensing pad 20, information on the urine and feces is transmitted to the state display unit 50, the state display unit 50 emits lights through the lamps 51a and 51b, and the speaker 53 emits a sound such that the manager who manages the patient can easily recognize the patient's diaper state through the visual and auditory effects. Moreover, the urine and feces state based on the sensed values of the humidity sensor 21b and 22b is displayed in graphs on the liquid crystal display 52 such that the manager can determine the patient's diaper state information in more detail.

The wall pad 60 is connected to the state display unit 50 via wireless communication and located on the wall of a patient room. The wall pad 60 comprises a microcontroller 62, a transceiver 64, a liquid crystal display 66, and a power source 68. The wall pad 60 displays identification information of a patient, from which urine and feces are sensed, including the patient's bed number, name, image, etc. through the liquid crystal display 66. By means of the wall pad 60, a medical team including a doctor, nurse, etc. can rapidly and easily identify the patient with urine and feces.

The transceiver 64 performs wireless communication with the transceiver 49 of the state display unit 50, and a virus piezoelectric element may preferably be used as the power source 68, like the power source 40 of the sensing pad 20. The transceiver 49 of the state display unit 50 and the transceiver 64 of the wall pad 60 transmit and receive information via wireless communication and may use ZigBee communication, for example, as shown in FIG. 2.

The multiport 70 is connected to the wall pad 60 via wire communication to gather the diaper state information through a serial communication line. For example, the wall pad 60 and the multiport 70 may perform communication using an RS485 serial communication network as shown in FIG. 2.

The server 75 is electrically connected to the multiport 70 through a USB and the like to calculate the medical history, treatment history, personal data, and changes in the frequency and amount of urine and feces of the patient and display the information in graphs through the image output unit 80, thus providing more detailed and practical information to the manager.

A program for managing the patients' urine and feces is stored in the server 75, and thus any changes in data due to the patients moving in and out of the hospital can be modified, resulting in efficient management.

As such, the existing sensing pads cannot accurately sense the urine and feces state; however, according to the present invention, accurate information with little or no margin of error about the urine and feces in the diaper worn by the patient is collected by the sensing pad provided with the temperature sensor, the humidity sensor, the hydrogen sensor, and the carbon dioxide sensor, which are configured to sense the gases generated in the intestines of the patient wearing the diaper, without having to use expensive sensors. The collected information is provided on the state display unit, and thus the production cost is reduced, resulting in high price competitiveness. Moreover, the management program operated on the server can be modified, and the image output unit allows the manager to determine the state of the diaper worn by the patient and the change or trend in urine and feces.

Furthermore, the reusable wear part 5 is mounted on the diaper worn by each of a plurality of patients in a public accommodation such as a hospital and connected to each state display unit 50 via wireless communication to display the collected information, information collected from a plurality of wear parts 5 is displayed on the wall pad 60 attached to the wall of a patient room by means of a short-range transmitter (ZigBee), state information of each diaper is gathered by the multiport 70 through a serial communication line, and the information collected by the multiport 70 including each patient's image, room, number of urine and feces, etc. is displayed on the image output unit 80 by means of the server 75 in which the patients' histories that can be modified are stored.

Therefore, it is possible to efficiently determine the state of each diaper worn by each of the plurality of patients and take urgent action based on the determined information such that the patient can be provided with a clean diaper at all times and the manager who manages the plurality of patients can easily, reliably and efficiently manage the patients, thus ensuring the ease of use.

It will be appreciated by those skilled in the art that the foregoing description is intended merely to illustrate the technical idea of the present invention, and various modifications, changes, and substitutions can be made without departing from the essential characteristics of the present invention. Accordingly, the embodiments disclosed in the present invention and the accompanying drawings are not used to limit, but to describe the technical idea of the present invention, and the scope of the technical idea of the present invention is not limited by these embodiments and the accompanying drawings. The protection scope of the present invention should be interpreted by the appended claims, and any technical ideas equivalent to the present invention should be interpreted to be included in the scope of the present invention.

### INDUSTRIAL APPLICABILITY

As described above, according to the present invention, the temperature sensor, the humidity sensor, the hydrogen sensor, and the carbon dioxide sensor are mounted in the sensing pad, which makes it possible to accurately sense the presence or absence of urine and feces, and the concentration sensor is further provided, which makes it possible to easily measure the amount of feces based on the amount of hydrogen and carbon dioxide.

Moreover, according to the present invention, the state display unit and the wall pad are connected via wireless communication, which makes it possible to facilitate the installation and management of the system, and the patient's name, image, change in the amount of feces, etc. are displayed on the wall pad such that the manager can rapidly and easily manage the patients. Furthermore, the program which can modify the input is operated on the server, which makes it possible to calculate the medical history, treatment history, personal data, and changes in the frequency and amount of urine and feces of the patient and display the information in graphs through the image output unit such that the patient can be provided with a clean diaper at all times and the manager can easily, reliably and efficiently manage the patients.

In addition, the virus piezoelectric element is used as the power source to drive the sensors and the microcontroller, which makes it possible to reduce the electricity cost and protect the environment.

1. A monitoring system for automatically alerting urine and feces of infants and critical patients, the monitoring system comprising:
a wear part which comprises a diaper cover into which a diaper is introduced and a sensing pad; and
a state display unit which is electrically connected to the sensing pad to receive and display information on a urine and feces state of the diaper sensed by the sensing pad,
wherein the sensing pad is inserted and fixed to the diaper cover and comprises a feces sensing means, a urine sensing means, and a microcontroller to sense the urine and feces state of the diaper when the top of the sensing pad comes into contact with the diaper,
wherein each of the feces sensing means and the urine sensing means comprises a hydrogen sensor, a carbon dioxide sensor, a humidity sensor, and a temperature sensor, wherein even when hydrogen and carbon dioxide are sensed by the hydrogen sensor and the carbon dioxide sensor, when heat and humidity are not sensed by the temperature sensor and the humidity sensor, the current state is determined to be a fart, not a feces state, thereby distinguishing between the feces state and the fart,
wherein the monitoring system further comprises a power source to drive the hydrogen sensor, the carbon dioxide sensor, the humidity sensor, and the temperature sensor, wherein the power source comprises a piezoelectric element in which viruses with helical proteins are stacked on the surface thereof,
wherein the sensing pad further comprises a concentration sensor which measures the amount of feces by measuring the amount of hydrogen and carbon dioxide sensed by the hydrogen sensor and the carbon dioxide sensor,
wherein the state display unit is mounted on a patient's bed, wherein the monitoring system further comprises a wall pad which is connected to the state display unit via wireless communication and located on the wall of a sickroom,
wherein the wall pad comprises a transceiver which performs wireless communication with a transceiver of the state display unit, a power source which comprises a piezoelectric element in which viruses with helical proteins are stacked on the surface thereof, and a liquid crystal display,
wherein the liquid crystal display of the wall pad displays identification information of a patient with urine and feces sensed from the wear part that can be reused in the diaper worn by each of a plurality of patients in a public accommodation comprising a hospital, including the patient's bed number, name, and image,
wherein the monitoring system further comprises a multiport, a server, and an image output unit, wherein the multiport is connected to the wall pad via wire communication to gather diaper state information through a serial communication line, wherein the server is electrically connected to the multiport to calculate the medical history, treatment history, personal data, and changes in the frequency and amount of urine and feces of a patient, from which urine and feces are sensed, by detecting the diaper state of the wear part worn by each of the plurality of patients, and display the information in graphs through the image output unit, and wherein a program for managing the patients' urine and feces is stored in the server such that the patients' information can be modified in the event of any changes in data due to the patients moving in and out of a hospital,
wherein information collected from the wear part is displayed on the wall pad, state information of each diaper is gathered by the multiport, and the information collected by the multiport including each patient's image, sickroom, and number of urine and feces is displayed on the image output unit by means of the server in which the patients' histories that can be modified are stored.
